(19) United States
(12) Patent Application Publication
(10) Pub. No.: US 2024/0299611 A1
(43) Pub. Date: Sep. 12, 2024

(54) VIRUS INACTIVATION SETUP FOR FLOWING FLUIDS

(71) Applicant: SMARTE TEKNOLOJI VE ENERJI SANAYI TICARET ANONIM SIRKETI, Istanbul (TR)

(72) Inventor: Melih BALLIKAYA, Istanbul (TR)

(73) Assignee: SMARTE TEKNOLOJI VE ENERJI SANAYI TICARET ANONIM SIRKETI, Istanbul (TR)

(21) Appl. No.: 18/281,353

(22) PCT Filed: Mar. 9, 2021
(86) PCT No.: PCT/TR2021/050210

§ 371 Date: Sep. 11, 2023

(51) Int. Cl. A61L 9/18; B01D 46/00; F24F 8/108; F24F 8/192

(52) U.S. Cl. CPC A61L 9/18; B01D 46/0028; F24F 8/108; F24F 8/192; A61L 2209/14; A61L 2209/16; B01D 2279/50

(57) ABSTRACT

A filtering device is provided. The filtering device includes two or more perforated plates opposing each other for allowing a fluid to flow through opposing perforating plates along a bulk fluid flow direction; the filtering device further includes a block sandwiched in-between the opposing perforated plates; the filtering device further includes one or more microwave emitting connector(s) arranged for provision of the block with microwaves at a frequency within the range between 1 GHz and 20 GHz, transverse to said intended fluid flow direction; wherein the block is provided with one or more fluid permeable pads disposed in-between the opposing perforated plates, said pads being formed from one or more materials with a dielectric constant within the range between 1 and 100.

### CROSS REFERENCE TO THE RELATED APPLICATIONS

This application is the national phase entry of International Application No. PCT/TR2021/050210, filed on Mar. 9, 2021, the entire contents of which are incorporated herein by reference.

### TECHNICAL FIELD

The present invention relates to an apparatus for inactivating viruses in flowing fluids. In particular, the present invention relates to inactivation of viruses using microwave.

### BACKGROUND

Disinfection by inactivation of viruses mainly includes the use of ultraviolet (UV) radiation, or chemical agents such as oxidants.

For inactivation of viruses in flowing fluids with the principles of chemistry, chemical agents inevitably mix to said fluid and get dragged along with the stream. Thus, the fluid gets contaminated. In the cases where the fluid is air or water to be directly contacted with mammals such as humans, the chemical agents should be separated from the fluid for elimination of possible harm. Such separation process brings extra and unmanageable costs. Disinfection by chemical agents take a high amount of time (e.g. around 150 minutes), and cannot be completed whilst the passage of a fluid through an apparatus dedicated to such disinfection without necessitating a long residence time. Hence, chemical means are not sufficiently suitable for inactivating viruses in flowing streams of fluids.

On the other hand, UV radiation itself is normally insufficient for an effective inactivation of viruses in flowing fluids, unless a tremendous amount of energy is consumed in the process. When the fluid is air, UV radiation causes the formation of ozone, which is harmful for living bodies. WO 2004/069288 A2 discloses the use microwaves as an alternative to UV radiation in sterilization of air. Air is passed through a wire mesh of a sterilization chamber. A vapor or a gas is adhered onto microorganisms to form a complex structure, and then said complex structure is subjected to high energy microwaves or UV radiation to cause a high electrostatic load on the complex structure, resulting in disinfection. Apparently, heating of the complex structure is also an important part of the related disinfection process.

US 2004/120 845 A1 relates to removal of pathogens in air circulated by HVAC systems. The system employs an ozone generator for preparing ozone to be mixed with water to obtain an oxidizing chemical agent. UV radiation is used in the formation of ozone from air oxygen. Microwaves are discussed as an alternative to UV radiation. Accordingly, said publication uses microwaves in production of ozone as a step in process for obtaining the oxidizing chemical agent.

It is assumed that in an indoors space, airborne viruses endure for days. In the case where the indoors space is air conditioned, the viruses are easily distributed all around the space, which aggravates a spread. Thus, in the process of normalization after a pandemic such as COVID-19, use of air conditioning systems in public indoor spaces (such as restaurants, workspaces or shopping malls) must be avoided, because air streaming from the air conditioning systems expedites the spread of the infection. Therefore, all of the advantages related to air conditioning shall remain unavailable until the pandemic is completely over.

Hence, inactivation of viruses in flowing fluids such as air or water is still an unsolved concern. This is especially the case when it comes to high fluid flow rates such as those air conditioning or ventilation systems, or systems for circulating swimming pools.

### OBJECTS OF THE INVENTION

The primary object of the present invention is to overcome the drawbacks outlined above.

Another object of the present invention is to propose an apparatus to be used in inactivation of viruses in streaming fluids.

A further object of the present invention is to propose an apparatus which is also easy to produce with low cost, which is easy to operate, which has long service life and easy to maintain by having a simple structure.

Other objects of the present invention will become apparent from accompanied drawings, brief descriptions of which follow in the next section as well as appended claims.

### SUMMARY

The apparatus proposed in the present application has a simple structure, which is easy to produce with low cost. The simple structure is also durable for a long service life, and easy to maintain. The apparatus does not consume high amounts of energy when in use, and is therefore economically advantageous in terms of operational costs. The disinfection process does not result in contamination of the processed fluid with harmful chemicals such as organic disinfectants or ozone.

The apparatus according to the present invention includes a block having a first side wall and a second side wall. The first side wall and second side wall oppose each other. The block is provided with at least two perforated plates opposing each other and sandwiching the block, arranged for allowing flow of fluids in an intended fluid flow direction transverse to a plane passing through both the first side wall and second side wall. The apparatus further comprises one or more microwave emitting connectors arranged for provision of the block with microwaves transverse to said intended fluid flow direction (D). The apparatus further includes one or more pads confined in the block, said pads being arranged to be permeable to both fluids and microwaves.

### BRIEF DESCRIPTION OF THE DRAWINGS

The appended drawings, brief description of which are provided below, are given solely for the purpose of exemplifying embodiments according to the present invention.

FIG. 1 shows a schematic view of a filtering device from an aspect parallel to an intended fluid flow direction.

FIG. 2 shows an exploded view of an exemplary filtering device according to the present invention.

FIG. 3A is exemplary detail showing a perspective view of pads.

FIG. 3B is Q-Q section view taken from FIG. 3A.

FIG. 3C is a side detail view of a filtering device according to the present invention.

FIG. 4A shows perspective view of a geometric example to an optional skeleton for being employed a substantially prismatic filtering device.

FIG. 4B shows perspective view of a geometric example to an optional skeleton for being employed a substantially cylindrical filtering device.

FIG. 5A shows a perspective view of an exemplary filtering device.

FIG. 5B shows a schematical T-T section view taken from FIG. 5A.

FIG. 6A shows an exploded view indicating such use of the filtering device according to the present invention.

FIG. 6B shows that a filtering device according to the present invention, a plurality of blocks can be arranged in series.

FIG. 7 shows a schematical view of an exemplary filtering device provided with a microwave receiving connector.

FIG. 8A shows a perspective view of an exemplary filtering device provided with a plurality of microwave receiving connectors.

FIG. 8B shows an upper view of an exemplary filtering device provided with a plurality of microwave receiving connectors.

### DETAILED DESCRIPTION OF THE EMBODIMENTS

Hereinafter, preferred embodiments of the present invention will be described in detail with reference to the accompanying drawings.

The present specification proposes a filtering device which effectively disinfects flowing fluid streams by inactivating viruses therein. The filtering device makes use of acoustic vibration, thereby causing resonance which mechanically damages outmost layers of viruses. Although the filtering device is effective in a wide variety of viruses (such as various types of Ebola, influenza, SARS, etc.) by easily arranging the frequency and energy density levels in accordance with a respective virus; the inactivation of spherical envelops is particularly easy due to their geometric limitations in terms of envelope surface area capable of holding the genetic material and enzymes in their inner volume. Thus, the inactivation occurs instantly (i.e. within a very short residence time) and with consumption of a very low amount of energy. In particular, disintegration of envelopes requires a minimum amount of energy and time, in inactivation of lipid enveloped viruses such as coronaviruses. Yet, advantages of the filtering device are not to be limited only to those related to coronaviruses, and it is possible to inactivate other viruses and even microorganisms which require a higher extent of acoustic vibration in disintegration thereof.

Within the context of the present application, the filtering device applies acoustic resonance to be exerted onto viruses in a fluid stream whilst passing through a block confined between perforated plates opposing each other.

Within the context of the present application, the term “microwave” refers to that with a frequency to induce acoustic vibration on an outermost layer of viruses, causing mechanical damage of such outermost layer. It is observed that virus species can be destroyed in this way, by defining and selecting microwave frequency ranges specific thereto. For instance, it is observed that frequencies within the range between 8.0 GHz and 9.5 GHz cause momentary deformations on envelopes of SARS-COV-2, resulting in rupture of said envelopes. Therefore, this range is considered suitable for being employed in damaging coronaviruses. When the frequency is within the narrower range between 8.2 GHz and 8.8 GHz, the efficiency in inactivation of SARS-COV-2 further increases, and a frequency around 8.4 GHz (i.e. within the range between 8.3 GHz and 8.5 GHZ) is considered sweet-spot in inactivation of such viruses in lowest time and with lowest energy consumption.

Since the present invention relies on mechanically damaging viruses by acoustic resonance, the filtering device (1) practically does not necessitate nor cause any heating of the fluid. Considering that heating would denature further biochemical/biological structures other than viruses, the filtering device (1) according to the present invention also enables a safe and effective sterilization of liquids such as vaccines, or bodily fluids such as blood samples. Therefore, the filtering device (1) according to the present invention and method of virus inactivation by using such filtering device is not to be limited for use in air or water disinfection, but also to in-vitro sterilization of other fluids with delicate organic or biological material which should be maintained, such as vaccines, blood samples and spinal fluid samples.

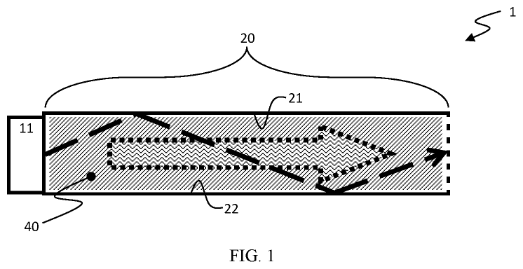

The filtering device (1) comprises two or more perforated plates (30) opposing each other, and a block (20) which can also be named as disinfection block, sandwiched in-between the opposing perforated plates (30). The block (20) can comprise a first side wall (21) and a second side wall (22) opposing said first side wall (21), arranged for allowing flow of fluids in an intended fluid flow direction (D) transverse to a plane passing through both the first side wall (21) and second side wall (22). An exemplary filtering device (1) is schematically depicted in FIG. 1, which shows the filtering device (1) from an aspect parallel to the intended fluid flow direction; the perforated plate (30) is not shown here for enabling the visualization of pad(s) (40), for emphasizing a microwaves advancement direction through the pad(s) (40) with a dotted bold arrow, and for showing possible reflections of microwaves on side walls (21 and 22) with a dashed arrow. The filtering device (1) further includes one or more microwave emitting connector(s) (11) arranged for provision of the block (20) with microwaves transverse to said intended fluid flow direction (D). The opposing perforated plates (30) preferably have electrically conductive surfaces facing each other, for reflecting microwaves. To this end, for instance:
a building material of the perforated plates (30) can be electrically conductive, such as by including one or more metals; or surfaces of perforated plates (30) opposing each other, that is, at the same time, facing the block (20) or one or more pads (40), can be coated, covered, or plated with one or more electrically conductive material, for instance said material can include one or more metals.

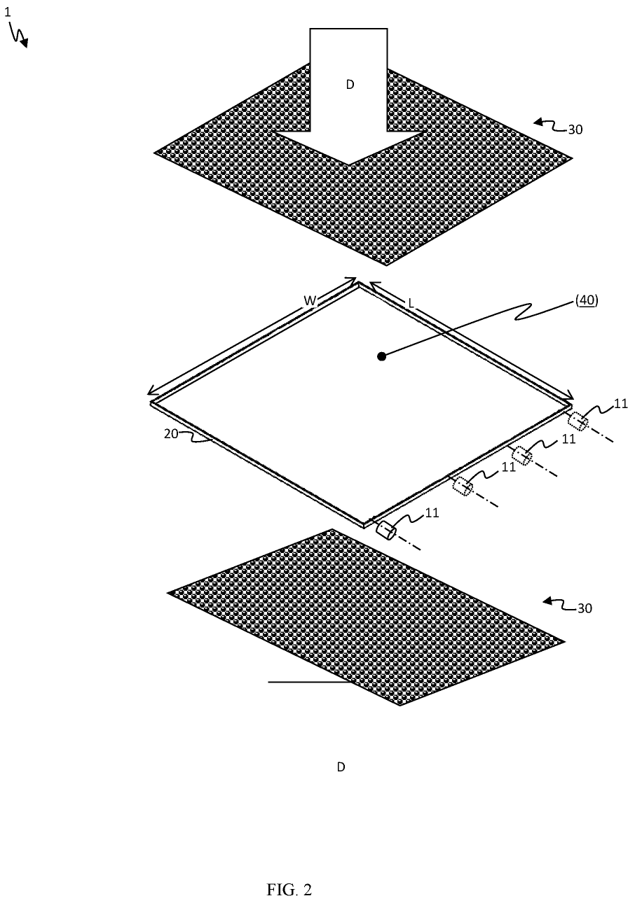

FIG. 2 shows an exploded view of an exemplary filtering device (1) according to the present invention. Perforations on the perforated plates (30) allow fluid flow in an intended bulk fluid flow direction (D), that is, in a general fluid flow direction. The perforated plates (30) are different from a wire mesh in that they inherently include a substantial extent of surface opposing the block (20), that is, said surface is substantially orthogonal to the intended fluid flow direction. This feature confines microwaves mainly within the block (20), simultaneously allowing a stream of fluid to be disinfected. In other words, the perforated plates (30) are arranged for minimization of any possible escape of microwaves away from the block (20) in directions having a component parallel to the intended fluid flow direction. The advantages attributed to perforated plates (30) apply to any embodiment of block (20) assemblies exemplified throughout the present specification, and to their obvious further variations.

As a further advantage, the fluid passing through the perforations would inevitably create eddy streams, resulting in an enhanced extent of mixing. This effect further enhances the uniformity in virus disinfection, because such movements due to eddy streams in the fluid causes an enhanced mixing, resulting in a uniform exertion of electromagnetic field throughout the fluid passing through the block (20).

The block (20) can be considered as a frame allow fluid passage therethrough. The block (20) can be in the form of an open frame such as it is visually exemplified in FIG. 1. Yet the block (20) is preferably in the form of a closed frame such as it is visually exemplified in the rest of the attached drawings. The form of closed frame enables confinement of a fluid stream therethrough, thus eliminates by-pass streams and enhances the effectiveness in fluid disinfection.

The microwave with a designated frequency can be thus applied into the block (20) via said one or more microwave emitting connector(s) (11), thereby causing acoustic resonance on the outermost layer of viruses in the passing fluid. For instance, in SARS-COV-2, the momentary envelope deformations acoustic resonance causes deviations from its original diameter (for instance, to extents calculable to be around 30 nm), resulting in inactivation due to mechanical damage in envelope structure. The same applies to other virus species by selection and easily replacing the one or more microwave emitting connector(s) (11) in accordance with another, frequency specific to another virus. Considering different sizes and outmost layer structures, the same principle applies to virus species other than SARS-COV-2, by employing respective specific frequencies selected from a range between 1 GHz and 20 GHz. Considering that currently SARS-COV-2 is the most urgent issue of the world, the frequency is preferably to be kept within the range between 8.0 GHz and 9.5 GHZ, more preferably between 8.2 GHz and 8.8 GHz, even more preferably between 8.3 GHz and 8.5 GHz. Considering that commercially available and low cost waveguides (such as WR90) can be used as microwave emitting connectors (11), the frequency can be arranged to be within the range between 6.5 GHz and 13 GHz which provides a single-mode provision of adjustable frequency, and within the range between 7 GHz and 12 GHz for an even higher accuracy. In rod-shaped (i.e. substantially cylindrical) viruses, 5.5-7 GHz provides a peak in resonance, and 20-25 GHz, 33-37 GHz and 45-50 GHz are also effective in inactivation of such viruses. On the other hand, a frequency range within 6-10 GHz is considered as an intersection applicable to inactivation of both spherical viruses and rod-shaped viruses. Hence, the filtering device (1) can be easily adapted in accordance with a specific frequency designated to inactivation of a target virus.

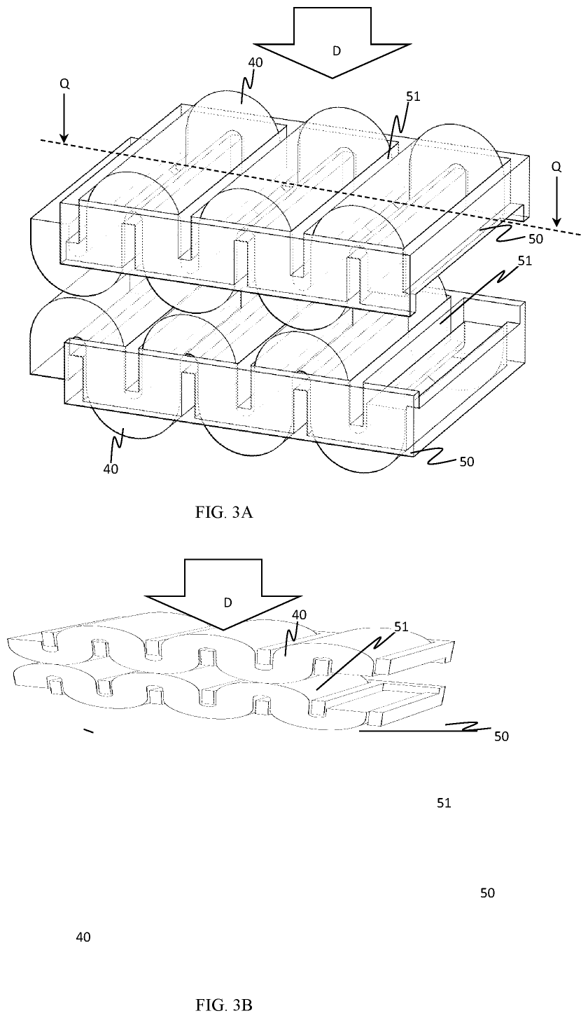

The block (20) is provided with one or more fluid permeable pads (40) disposed in-between the opposing perforated plates (30). The pads (40) provide fluid passages, that is, through holes; and to provide surfaces for retention of particles such as microorganisms and viruses that may be present in the fluid flowing through the pads (40). An exemplary detail showing a perspective view of pads (40) is showing in FIG. 3A. Here, the pads (40) are preferred to be mechanically stabilized on a skeleton (50) which includes a plurality of consecutive bars (51) parallel to each other disposed on a plane in common with each other, arranged to support said one or more pads (40) in alternating sides of said pads (40). FIG. 3B shows Q-Q section view taken from FIG. 3A to emphasize the example to how consecutive bars (51) can be used in supporting the pads (40) in alternating sides of said pads (40). FIG. 3C is a side detail view of a filtering device according to the present invention, showing an exemplary sinusoidal packing of pads (40) using consecutive respective skeletons (50) along the intended bulk fluid flow direction (D). As shown in FIG. 3A to FIG. 3C, in particular in FIG. 3C, the filtering device (1) can include a plurality of pads (40) along the intended bulk fluid flow direction (D).

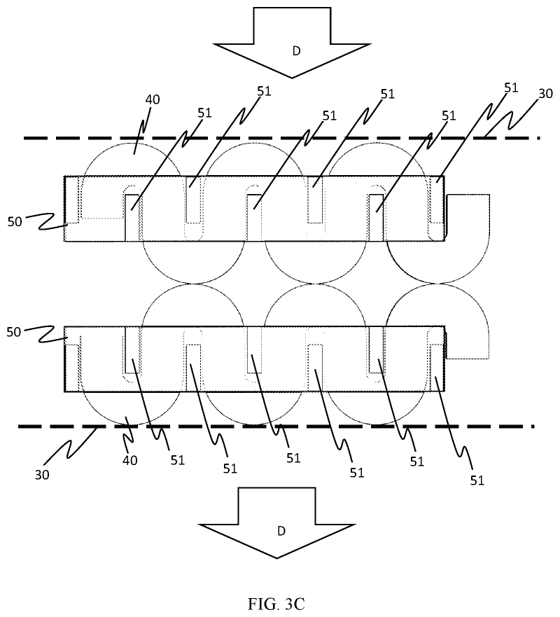

The filtering device (1) can be designed in various geometries. For instance, FIG. 4A and FIG. 4B implicate geometric examples to optional skeletons (50) which can be preferred to be employed in filtering devices (1) in geometries such as a prismatic geometry and a cylindrical geometry, respectively.

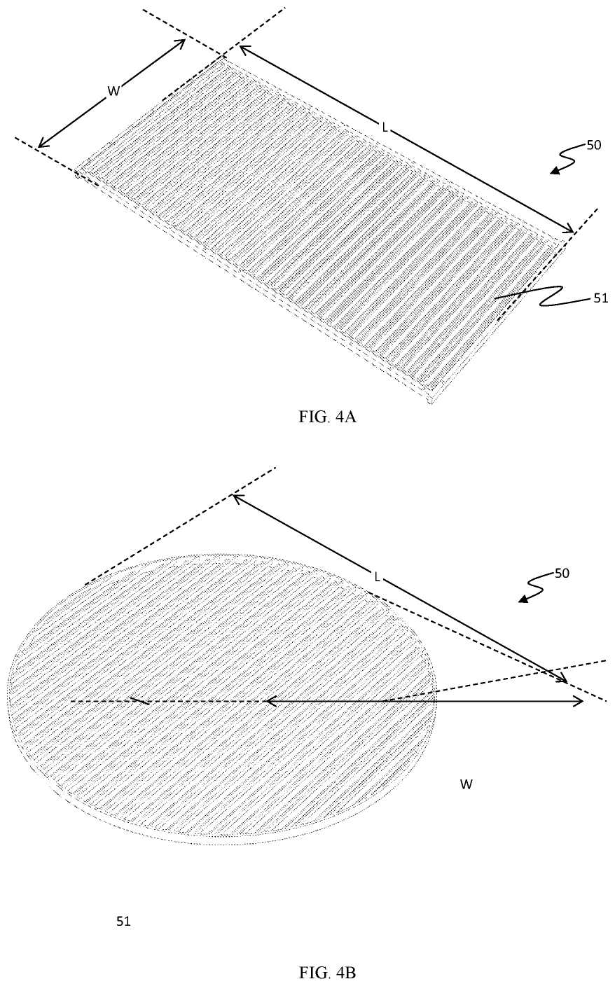

FIG. 5A shows a perspective view of an exemplary filtering device (1) visualizing that the intended bulk fluid flow direction (D) can be substantially orthogonal or transverse to one or more of the perforated plate(s) (30). FIG. 5B shows a schematical T-T section view from an aspect ratio orthogonal to the perforated plate(s) (30) showing the advancement of microwaves (bold dotted arrow) along the block (20), and here, transverse to the intended bulk fluid flow direction (D).

The pads (40) can include a HEPA (abbreviation of: high-efficiency particulate air, also known as high-efficiency particulate absorbing and high-efficiency particulate arrestance) filter, and/or an artificial sponge, and/or a twisted fibrous structure which can be substantially formed from a plurality of elongate pieces of fibers twisted together.

In the case where the pads (40) include such twisted fibrous structure, the fibers provide non-linear fluid passages, that is, through holes for meandering the flow trajectory, to the fluid streaming between the perforated plates.

The pads (40), for instance said fibers, are formed from one or more materials with a dielectric constant low enough to permit microwaves to penetrate therethrough without being completely absorbed by said materials. To this end, said materials can have a dielectric constant of up to 100. Preferably, said materials can have a dielectric constant of up to 10, thereby use of a minimalized number of microwave emitting connectors (11) can be used for achieving virus inactivation or disinfection. More preferably, said materials can have a dielectric constant of up to 6, thereby a minimalized energy consumption by the microwave emitting connectors (11) can be sufficient for achieving virus inactivation or disinfection even with a high PPI value in the pads (40).

For instance, the one or more materials of which the one or more pads (40) is formed from, e.g. the fibers, can be synthetic and substantially formed from one or more polymeric materials, which can be for instance selected from polyurethane (PU), polyethylene (PE), polypropylene (PP), polyamide, polybutylene terephthalate (PBT), polyester, phenol-formaldehyde (PF), polyvinyl chloride fiber (PVC), acrylic polyesters, pure polyester PAN fibers, carbon fibers, aromatic polyamides, and mixtures thereof. Preferably, said one or more polymeric materials are selected from thermosetting polymers, so that in case of a temperature increase, the pads (40) are protected from a temperature-related deformation.

Within the context of the present application, the term “fiber” can also be spelled as “fibre”.

The pads (40) can be provided in the form of one or more blankets, which are plied or brought into an undulated shape as visualized in FIG. 3A to FIG. 3C. For ease of montage, a skeleton (50) which includes a plurality of consecutive bars (51) parallel to each other disposed on a plane in common with each other, can be provided with such one or more blankets of pads (40). Such skeleton (50) having consecutive bars (51) supporting said pads (40) in alternating sides of said pads (40), provide a stabilized undulated configuration to said pads (40). Thus, an increased amount of undulated pads (40) can be prepared in a substantially flat filter configuration supported by the skeleton, to be placed in-between the perforated plates (30).

The pads (40) cause micro-deviations, in particular when the pads (40) include said twisted fibrous structure, causes local deviations in fluid flow direction when passing therethrough, which correspond to micro-turbulences, enhancing the uniformity of virus inactivation or disinfection throughout the block (20).

Thanks to said local deviations, the flow path of the fluid is increased when passing through the block (20) when compared to a vertical distance between the opposing perforated plates (30); substantially increasing the probability of retention of the particles (here: viruses and microorganisms) on surfaces of the pads (40). The particles when retained on said surfaces are thus exposed to a higher extent of microwave energy when compared to a block (20) configuration without pads (40) at which the particles would be passed through the opposing perforated plates (30) without being retained on surfaces of any pads (40). As a result, the filtering effect of the pads (40) enhances the disinfection or virus inactivation performance of the filtering device (1).

### Example 1

Opposing perforated plates (30) are employed each of which have dimensions of 25 cm×30 cm, which corresponds to a 750 cm² footprint area orthogonal to a general, bulk fluid flow direction.

The fluid passed through the opposing perforated plates (30) was air at 23° C. and 54% relative humidity; in accordance with ISO 15714 standard method, at a flow rate of 2.3 m/s corresponding to 621 cubic meters per hour. A static pressure loss between the perforated plates is measured as being not more than 20 Pa, so no dramatic extent of pressure drop has been observed. The pressure drop was considered to be insignificant.

Madin-Darby Canine Kidney (MDCK) cells were placed into a 5 wt. % newborn calf serum (NCS) enriched cell growth media, and the resulting mixture was then placed into 96-wells plates. As an example, 10\hat{ }\hat{ }7 TCID₅₀ of human H1N1 virus were diluted to obtain a virus contaminated mixture of 10\hat{ }\hat{ }5 TCID₅₀ and then passed through the block (20) in accordance with the following procedure:
Step 1. Using a nebulizator, 4 milliliters of the virus contaminated mixture was passed through a gel membrane at an air flow exit side of the block (20) which is not yet provided with the pads (40). Samples were collected from the gel membrane.
Step 2. For comparison with the results of the above step 1: using the nebulizator, 4 milliliters of the virus contaminated mixture were passed through a gel membrane equivalent to that used in the step 1, at an air flow exit side of the block (20) which is provided with the pads (40). Samples were collected from the gel membrane.
Step 3. Radio frequency was continued to be emitted into the block (20) for a further 45 minutes. Then, swabbing samples were collected through each perforation on the perforated plates (30) to be suspended in sterile phosphate-buffered saline (PBS).

### Example 2

For the case of H1N1 virus at a concentration of 10\hat{ }10 viruses per cubic meter of air, a virus inactivation rate of no less than 99% was achieved.

Each of the HEPA filter, and/or an artificial sponge, and/or a twisted fibrous structure, can be considered as porous media. For instance, for the twisted fibrous structure, the non-linear fluid passages can be considered to correspond to pores. There are two methods to describe porosity of porous media:
1. Porosity which is a ratio between volume of voids and total volume of the medium.
2. PPI (Pores Per Inch) which is the number of pores in one linear inch.

Open cell, reticulated, commercially available Polyurethane foams in the form of a plurality of elongate pieces of fibers twisted together, is an exemplary material for filtering unwanted particulate from the air, for being used as a porous medium within the context of the present invention. The fluid passages can randomly have polygonal (e.g. pentagonal, dodecahedron, etc.) geometric shapes that can be controlled in respective manufacturing processes of the pads (40) and may be varied to meet specific applications. These shapes are referred to as pores. The term porosity, usually measured in PPI (Pores Per Inch) designates the number of pores in one linear inch. Counting the pores is a visual indication of porosity which directly coincides with an airflow tested measurement that defines a range of airflow for a given porosity. In general, with thickness along a bulk, general flow direction being equal, the higher the porosity, the more pressure it takes to pull or push air through the foam filter media, which corresponds to a static pressure drop between sides of the pads (40) facing the opposing perforated plates (30). For example, a 10 PPI foam can be considered to have relatively large pores, approximately 0.10″ in diameter and offers very little resistance to airflow. Consequently, it is not very effective at capturing small particles and arrestance values are typically below 50%. By comparison, an 60 PPI foam, has relatively small pores, approximately 0.015″ diameter. Resistance to airflow can be quite high when thickness of the pads (40) is greater than 0.50″. However, arrestance values can reach well into the 90% range. Even at rather low porosities such as 10 PPI, the pathogen load in the air flowing through the pads (40) is observed to be dramatically decreased by arrestance, that is the microorganisms or viruses being retained on surfaces of the pads (40). The higher the porosity, the decrease in pathogen load is even better than those available with low porosities.

It is observed that 90% of pathogen load decrease (=virus inactivation rate) is easily availed with any embodiment of the filtering device according to the present invention, notwithstanding the extent of porosity, based on experiments made in accordance with ISO 15714 standard.

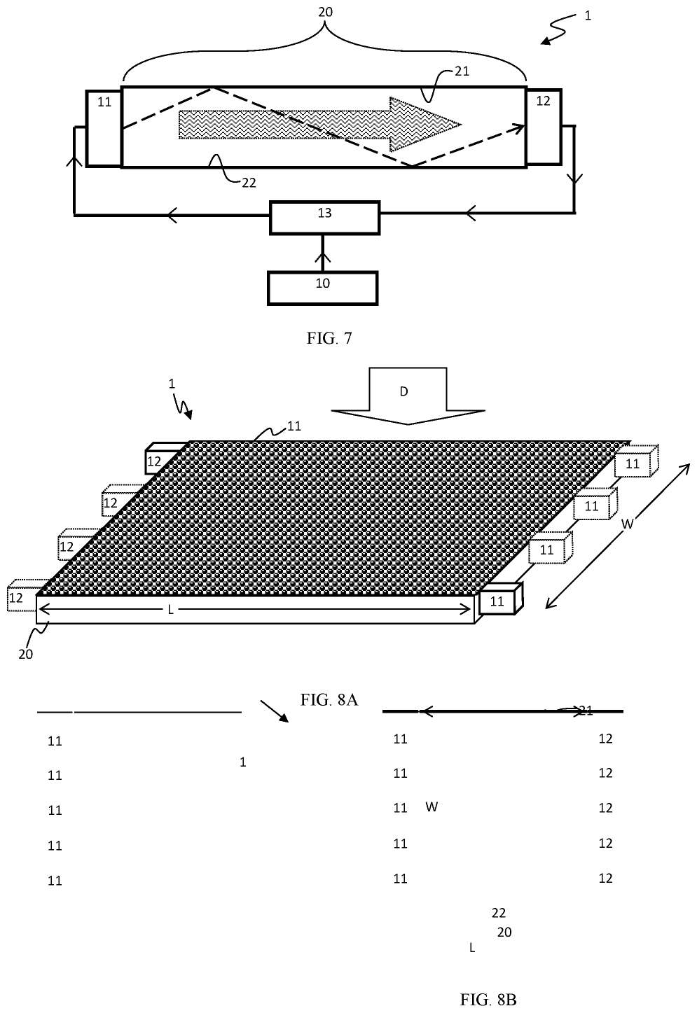

Preferably, such plurality of blocks (20) can be arranged in series; i.e. such that the fluid flows/passes through the plurality of consecutively arranged blocks (20). Preferably, the plurality of blocks (20) can be confined between perforated plates (30) arranged to allow said flow of the fluid through the blocks (20). This measure at least partly blocks the escape of microwave away from the plurality of blocks (20). Even more preferably, perforated plates (30) can be sandwiched between consecutive blocks (20). Even more preferably, each block (20) can be confined between perforated plates (30), such that a perforated plate (30) is sandwiched between each pair of consecutive blocks (20) throughout the intended flow direction. This measure enhances the uniformity in distribution of microwaves throughout the plurality of blocks (20), and enhances the accuracy and precision in disinfection. In FIG. 8A and FIG. 8B an exemplary way of how the fluid flow port can be in fluid communication with a propelling means is depicted, to effect the flow of a respective fluid in a fluid flow direction transverse to an area to be scanned which may be defined by a width (W) and a length (L) of the block (20), said width (W) and length (L) defining a plane which can be substantially parallel to one or more of said perforated plates (30).

In the case where the streaming fluid is air, when in use, the block (20) can be named as a “vironance airflow cell”.

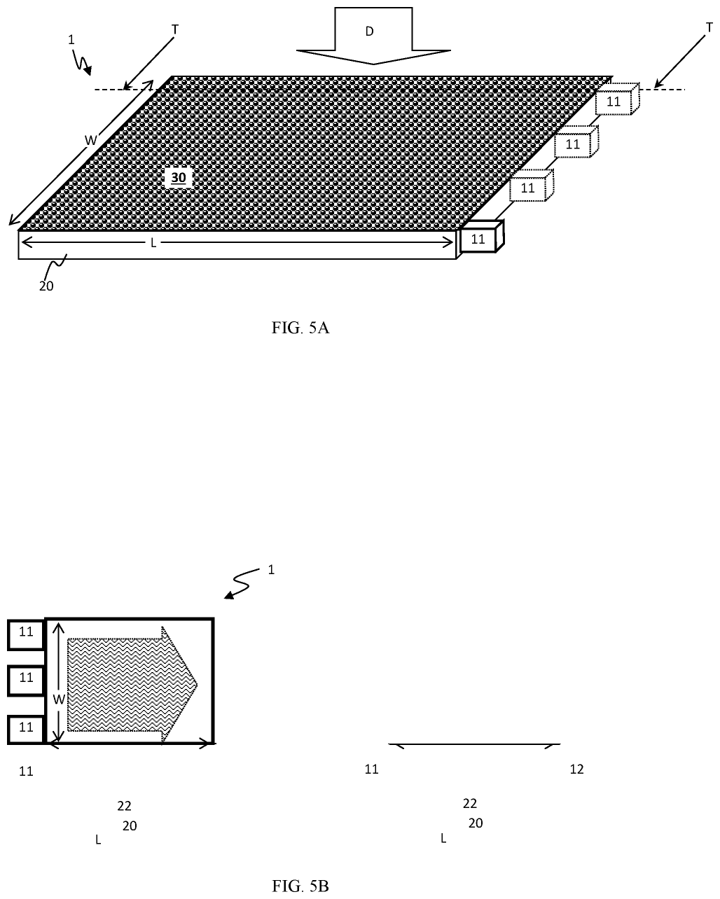

The filtering device (1) according to the present invention can be integrated to a port (60) for fluid flow to/from a pressurizing means (61) of a heating, ventilation and/or air conditioning system. FIG. 6A shows an exploded view indicating such use of the filtering device (1) according to the present invention. FIG. 6B shows that shows that in a filtering device (1) according to the present invention, a plurality of blocks (20) can be arranged in series. A holding means (62) with one or more air flow openings (63) can be employed for aligning and securing one or more filtering devices (1), for example, to a port (60).

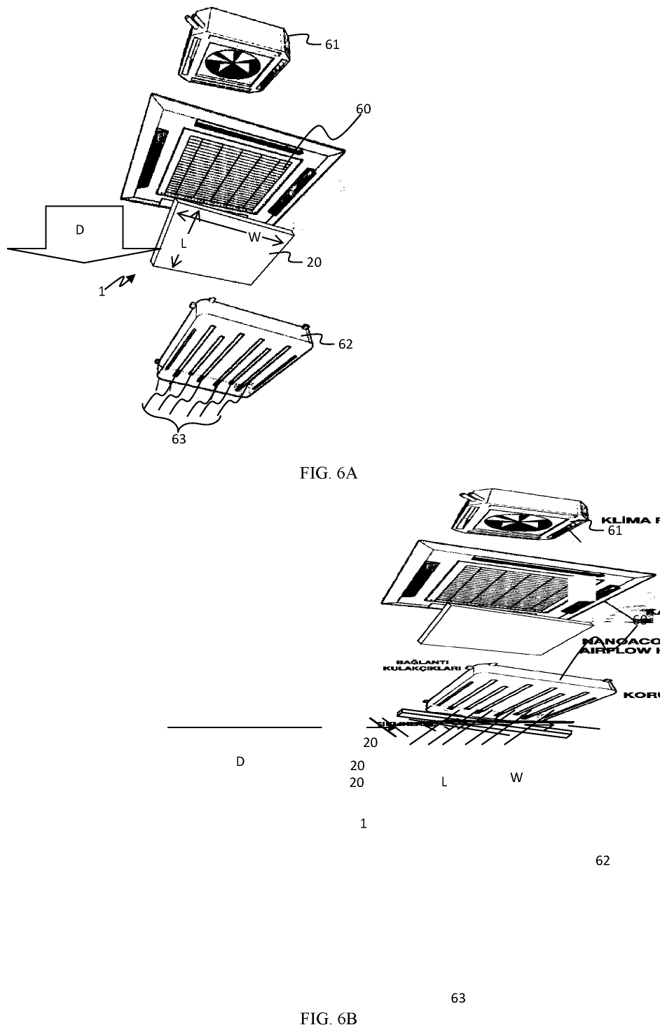

The filtering device (1) can preferably be further provided with one or more microwave receiving connectors (12), arranged to receive/collect microwave beams released from the microwave emitting connector (11) into the block (20), e.g. upon such microwave beams travelling through the block (20). Thus, microwaves travelled along the block (20) are collected by the microwave receiving connectors (12). FIG. 7, FIG. 8A and FIG. 8B respectively show a schematical view, a perspective view, and an upper view of an example to such filtering device (1). The exemplary filtering device in FIG. 7 is provided with a single microwave receiving connector (12); whereas the exemplary filtering devices in FIG. 8A and FIG. 8B are provided with a plurality of microwave receiving connectors (12).

As visually exemplified in FIG. 7, in which the filtering device (1) is shown from an aspect parallel to the intended fluid flow direction, the microwaves (i.e. their remaining energy) collected by the microwave receiving connector(s) (12) can be conducted to one or more circulator(s) (13) (within the present context, one or more RF circulator(s) or microwave circulator(s)) arranged to further conduct/feed the collected microwaves to the microwave emitting connector(s) (11). Accordingly, the filtering device (1) can be further provided with one or more circulator(s) (13) for at least partial recovery of the microwave energy from the microwave receiving connectors (12) to supply the microwave emitting connector (11). A difference between microwave energy extents at the microwave emitting connectors (11) and the microwave receiving connectors (12) can be replenished by one or more power source(s) (10) (within the present context, one or more RF power source(s) or microwave power source(s)) in communication with the microwave circulator (13).

### REFERENCE SIGNS

1 filtering device
10 power source
11 microwave emitting connector
12 microwave receiving connector
13 circulator
20 block
21 first side wall
22 second side wall
30 perforated plate
40 pad
50 skeleton
51 bar
60 port
61 pressurizing means
62 holding means
63 opening
100 apparatus
D intended bulk air flow direction or general air flow direction
L length
W width

What is claimed is:

1. A filtering device comprising at least two perforated plates opposing each other for allowing a fluid to flow through opposing perforated plates along a bulk fluid flow direction;
wherein the filtering device further comprises a block sandwiched in-between the opposing perforated plates;
wherein the filtering device further comprises at least one microwave emitting connector arranged for provision of the block with microwaves at a frequency within a range between 1 GHz and 50 GHz, transverse to the intended fluid flow direction;
wherein the block is provided with at least one fluid permeable pad disposed in-between the opposing perforated plates, the at least one fluid permeable pad being formed from at least one material with a dielectric constant within a range between 1 and 100.

2. The filtering device according to claim 1, wherein the opposing perforated plates have electrically conductive surfaces facing each other.

3. The filtering device according to claim 1, wherein the at least one material has a dielectric constant of up to 10 or 6.

4. The filtering device according to claim 3, wherein the at least one fluid permeable pad comprises at least one high-efficiency particulate arrestance filter, and/or at least one artificial sponge, and/or at least one twisted fibrous structure.

5. The filtering device according to claim 4, wherein the at least one fluid permeable pad comprises at least one twisted fibrous structure formed from a plurality of elongate pieces of fibers twisted together.

6. The filtering device according to any of the claim 1, wherein at least one material of which the at least one fluid permeable pad is formed from, is selected from the group consisting of polyurethane, polyethylene, polypropylene, polyamide, polybutylene terephthalate, polyester, phenol-formaldehyde, polyvinyl chloride fiber, acrylic polyesters, pure polyester PAN fibers, carbon fibers, aromatic polyamides, and mixtures polyurethane, polyethylene, polypropylene, polyamide, polybutylene terephthalate, polyester, phenol-formaldehyde, polyvinyl chloride fiber, acrylic polyesters, pure polyester PAN fibers, carbon fibers, and aromatic polyamides.

7. The filtering device according to claim 1, wherein the at least one fluid permeable pad is in a form of at least one blanket plied into an undulated shape.

8. The filtering device according to claim 7, comprising a skeleton, wherein the skeleton comprises a plurality of consecutive bars parallel to each other disposed on a plane in common with each other, arranged to support the at least one fluid permeable pad in alternating sides of the at least one fluid permeable pad.

9. The filtering device according to claim 1, wherein the filtering device is further provided with at least one microwave receiving connector, arranged to collect at least one microwave beam released from the at least one microwave emitting connector into the block, upon the at least one microwave beam travelling through the block.

10. The filtering device according to claim 1, wherein the at least one microwave emitting connector is arranged for provision of frequencies within a range between 1 GHz and 20 GHz.

11. The filtering device according to claim 10, wherein the at least one microwave emitting connector is arranged for provision of frequencies within a range between 6.5 GHz and 13 GHz, between 7 GHz and 12 GHz, or between 5.5 GHz and 7 GHz.

12. The filtering device according to claim 10, wherein the at least one microwave emitting connector is arranged for provision of frequencies within a range between 1 GHz and 8.8 GHz or between 8.3 and 8.8 GHz.

13. The filtering device according to claim 12, wherein the at least one microwave emitting connector is arranged for provision of frequencies within a range between 8.3 GHz and 8.5 GHz.

14. The filtering device according to claim 1, wherein the at least one microwave emitting connector is arranged for provision of frequencies within a range between 20 GHz and 25 GHZ, or between 33 GHz and 37 GHz, or between 45 GHz and 50 GHz.

15. A heating, ventilation and/or air conditioning system comprising the filtering device according to claim 1.

16. The filtering device according to claim 2, wherein the at least one material has a dielectric constant of up to 10 or 6.

17. The filtering device according to claim 2, wherein at least one material of which the at least one fluid permeable pad is formed from, is selected from the group consisting of polyurethane, polyethylene, polypropylene, polyamide, polybutylene terephthalate, polyester, phenol-formaldehyde, polyvinyl chloride fiber, acrylic polyesters, pure polyester PAN fibers, carbon fibers, aromatic polyamides, and mixtures of polyurethane, polyethylene, polypropylene, polyamide, polybutylene terephthalate, polyester, phenol-formaldehyde, polyvinyl chloride fiber, acrylic polyesters, pure polyester PAN fibers, carbon fibers, and aromatic polyamides.

18. The filtering device according to claim 3, wherein at least one material of which the at least one fluid permeable pad is formed from, is selected from the group consisting of polyurethane, polyethylene, polypropylene, polyamide, polybutylene terephthalate, polyester, phenol-formaldehyde, polyvinyl chloride fiber, acrylic polyesters, pure polyester PAN fibers, carbon fibers, aromatic polyamides, and mixtures of polyurethane, polyethylene, polypropylene, polyamide, polybutylene terephthalate, polyester, phenol-formaldehyde, polyvinyl chloride fiber, acrylic polyesters, pure polyester PAN fibers, carbon fibers, and aromatic polyamides.

19. The filtering device according to claim 4, wherein at least one material of which the at least one fluid permeable pad is formed from, is selected from the group consisting of polyurethane, polyethylene, polypropylene, polyamide, polybutylene terephthalate, polyester, phenol-formaldehyde, polyvinyl chloride fiber, acrylic polyesters, pure polyester PAN fibers, carbon fibers, aromatic polyamides, and mixtures of polyurethane, polyethylene, polypropylene, polyamide, polybutylene terephthalate, polyester, phenol-formaldehyde, polyvinyl chloride fiber, acrylic polyesters, pure polyester PAN fibers, carbon fibers, and aromatic polyamides.

20. The filtering device according to claim 5, wherein at least one material of which the at least one fluid permeable pad is formed from, is selected from the group consisting of polyurethane, polyethylene, polypropylene, polyamide, polybutylene terephthalate, polyester, phenol-formaldehyde, polyvinyl chloride fiber, acrylic polyesters, pure polyester PAN fibers, carbon fibers, aromatic polyamides, and mixtures of polyurethane, polyethylene, polypropylene, polyamide, polybutylene terephthalate, polyester, phenol-formaldehyde, polyvinyl chloride fiber, acrylic polyesters, pure polyester PAN fibers, carbon fibers, and aromatic polyamides.
